(19) United States
(12) Patent Application Publication
(10) Pub. No.: US 2025/0195169 A1
(43) Pub. Date: Jun. 19, 2025

(54) SENSING DEVICE, SYSTEM AND METHOD FOR SURGICAL SUTURE THREAD

(71) Applicant: METAL INDUSTRIES RESEARCH & DEVELOPMENT CENTRE, Kaohsiung (TW)

(72) Inventors: Yu-Sung Hsieh, Tainan City (TW); Wei-Hung Shih, Kaohsiung City (TW); Bing-Feng Huang, Kaohsiung City (TW)

(21) Appl. No.: 18/539,762

(22) Filed: Dec. 14, 2023

(51) Int. Cl. A61B 90/00; G01L 5/04

(52) U.S. Cl. CPC A61B 90/06; G01L 5/042; G01L 5/047; A61B 2090/065

(57) ABSTRACT

A sensing device is provided to be planted onto a bone to sense a force applied to a soft tissue by a surgical suture thread and includes a carrier and a wireless sensor mounted in a through hole of the carrier. The wireless sensor can be contacted by the surgical suture thread passed through the through hole and activated by a wireless reading device to generate a resonance frequency signal. The resonance frequency signal can be converted to a pressure value to represent the force applied to the soft tissue by the surgical suture thread.

### FIELD OF THE INVENTION

This invention relates to sensing device, system and method which are provided to confirm a pulling force of a surgical suture thread applied to a soft tissue during or after surgery.

### BACKGROUND OF THE INVENTION

Surgical suturing is a common medical treatment, for example, tendon suturing is one of common orthopedics surgeries. However, the force applied to a surgical suture thread cannot be known during surgery, and doctor usually determines whether the force applied to the surgical suture thread is appropriate (or not) according to his/her practical experiences only. If the force applied to the surgical suture thread by the doctor is too tight or too loose during surgery, patient may feel uncomfortable after surgery.

### SUMMARY

One object of the present invention is to provide a device, a system and a method for sensing a surgical suture thread, which are provided to sense a force applied to a soft tissue by the surgical suture thread during or after surgery.

A sensing device disclosed in the present invention is provided to be planted on a bone to sense a tension in a surgical suture thread. The sensing device includes a carrier and a wireless sensor which is mounted in a through hole of the carrier. While the surgical suture thread is inserted into and passed through the through hole, the wireless sensor is contacted by the surgical suture thread and activated by a wireless reading device to generate a resonance frequency signal.

A sensing system for surgical suture thread of the present invention includes a plurality of sensing devices mentioned above and a wireless reading device. A resonance frequency range of the wireless sensor of each of the sensing devices is different and not overlapping. The wireless reading device includes a controller, an inductive coil, a reader, and a computer. The controller is provided to generate and transmit oscillation signals to the inductive coil. Each of the oscillation signals has an oscillation frequency range not overlapping with others and includes oscillation sub-signals with different oscillation frequencies. The oscillation sub-signals are provided to be transmitted to the inductive coil to active the wireless sensor of each of the sensing devices. While the oscillation frequency of one of the oscillation sub-signals of one of the oscillation signals is consistent with one of resonance frequencies within the resonance frequency range of the wireless sensor of one of the sensing devices, a resonance frequency signal is generated by the wireless sensor. The reader is provided to read a phase value corresponding to the resonance frequency signal, and the computer is provided to receive and convert the phase value to a pressure signal.

A sensing method disclosed in the present invention is provided to sense a force applied to a soft tissue by a surgical suture thread. The sensing method includes the steps of providing a first sensing device and a second sensing device which are mounted on a bone, providing a wireless reading device, and activating the first sensing device and/or the second sensing device. The first sensing device includes a first carrier and a first wireless sensor mounted in a first through hole of the first carrier, and the second sensing device includes a second carrier and a second wireless sensor mounted in a second through hole of the second carrier. A first resonance frequency range of the first wireless sensor is not overlapping with a second resonance frequency range of the second wireless sensor. The first wireless sensor can be contacted by the surgical suture thread passed through the first through hole to generate a first resonance frequency within the first resonance frequency range, and the second wireless sensor can be contacted by the surgical suture thread passed through the second through hole to generate a second resonance frequency within the second resonance frequency range. The wireless reading device includes a controller, an inductive coil, a reader, and a computer. The controller generates a first oscillation signal with a first oscillation frequency range and a second oscillation signal with a second oscillation frequency range which is not overlapping with the first oscillation frequency range. The first oscillation signal includes multiple first oscillation sub-signals with different oscillation frequencies, and the second oscillation signal includes multiple second oscillation sub-signals with different oscillation frequencies. Activation of the first or second sensing device includes six steps as follows. In a first step, the first sensing device is activated by the first oscillation sub-signals, and the first wireless sensor in the first sensing device generates a first resonance frequency signal while the oscillation frequency of one of the first oscillation sub-signals is consistent with the first resonance frequency of the first wireless sensor. If the oscillation frequency of each of the first oscillation sub-signals is not consistent with the first resonance frequency of the first wireless sensor, the first sensing device is activated by the second oscillation sub-signals, and the first wireless sensor in the first sensing device generates the first resonance frequency signal while the oscillation frequency of one of the second oscillation sub-signals is consistent with the first resonance frequency of the first wireless sensor. The reader reads a first phase value corresponding to the first resonance frequency signal in a second step. The first phase value is converted to a first pressure signal by the computer, and a first pressure value of the first pressure signal is shown on a display in a third step. In a fourth step, the second sensing device is activated by the second oscillation sub-signals if the oscillation frequency of one of the first oscillation sub-signals is consistent with the first resonance frequency of the first wireless sensor. The second wireless sensor of the second sensing device activated by the second oscillation sub-signals generates a second resonance frequency signal while the oscillation frequency of one of the second oscillation sub-signals is consistent with the second resonance frequency of the second wireless sensor. In the fourth step, the second sensing device is activated by the first oscillation sub-signals if the oscillation frequency of one of the second oscillation sub-signals is consistent with the first resonance frequency of the first wireless sensor. The second wireless sensor of the second sensing device activated by the first oscillation sub-signals generates the second resonance frequency signal while the oscillation frequency of one of the first oscillation sub-signals is consistent with the second resonance frequency of the second wireless sensor. The reader reads a second phase value corresponding to the second resonance frequency signal in a fifth step. And the second phase value is converted to a second pressure signal by the computer and a second pressure value of the second pressure signal is shown on the display in a sixth step.

The wireless reading device of the present invention can sense the pressure value applied on the wireless sensor mounted in the through hole by the surgical suture thread and can confirm whether the force applied to the surgical suture thread is proper according to the pressure value.

### DESCRIPTION OF THE DRAWINGS

FIG. 1 is a perspective exploded diagram illustrating a sensing device in accordance with a one embodiment of the present invention.

FIG. 2 is a perspective assembly diagram illustrating a sensing device in accordance with a one embodiment of the present invention.

FIG. 3 is a partial sectional view of a sensing device and a surgical suture thread in accordance with one embodiment of the present invention.

FIGS. 4 to 6 are diagrams illustrating a sensing device and a wireless reading device in accordance with one embodiment of the present invention.

FIG. 7 is a flowchart illustrating steps of activating a first sensing device and/or a second sensing device in accordance with one embodiment of the present invention.

### DETAILED DESCRIPTION OF THE INVENTION

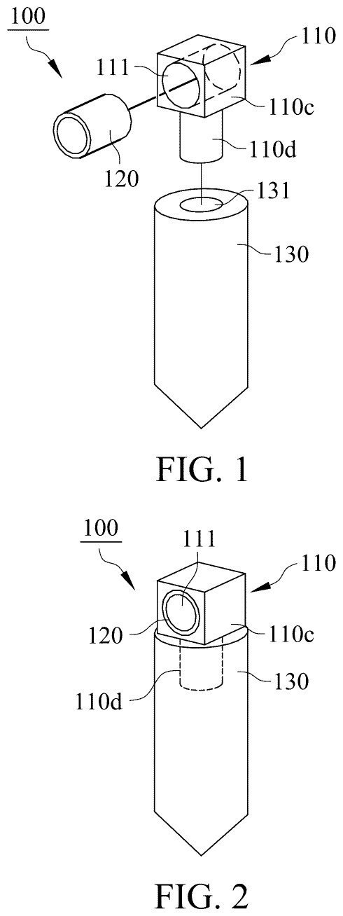

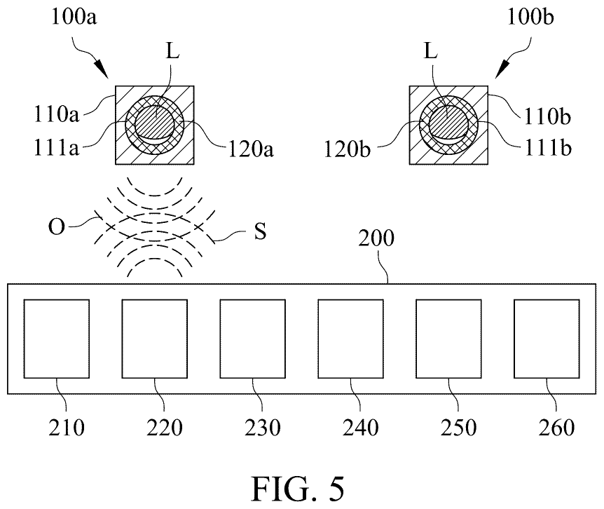

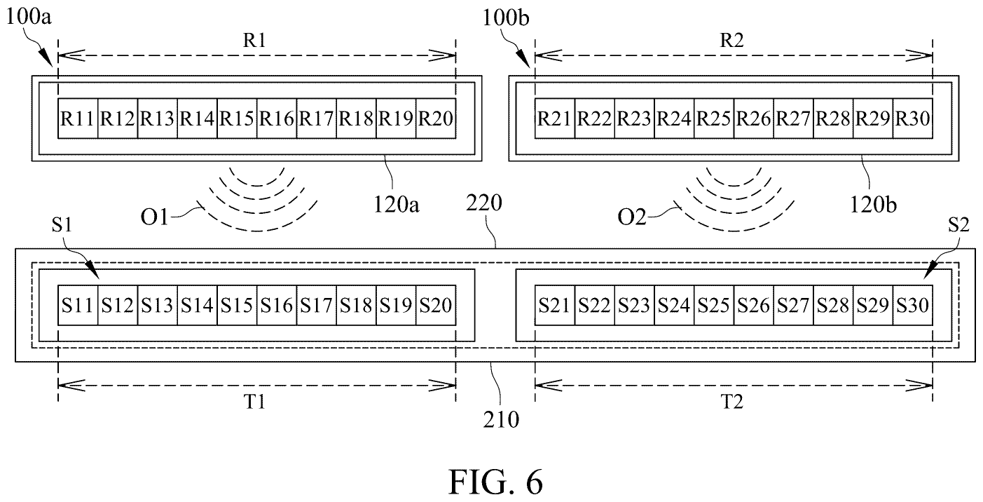

With reference to FIGS. 1 to 3, the present invention discloses a sensing device 100 which can be planted onto a bone (not shown) to sense a pulling force applied to a surgical suture thread L for soft tissue suturing (e.g. tendon suturing). The sensing device 100 for surgical suture thread includes a carrier 110 having a through hole 111 and a wireless sensor 120 which is but not limit to an inductor-capacitor (LC) passive pressure sensor. The wireless sensor 120 is mounted in the through hole 111, and the surgical suture thread L can contact the wireless sensor 120 while it is inserted into and passed through the through hole 111. As shown in FIG. 4, the wireless sensor 120 can be activated by a wireless reading device 200 to generate a resonance frequency signal.

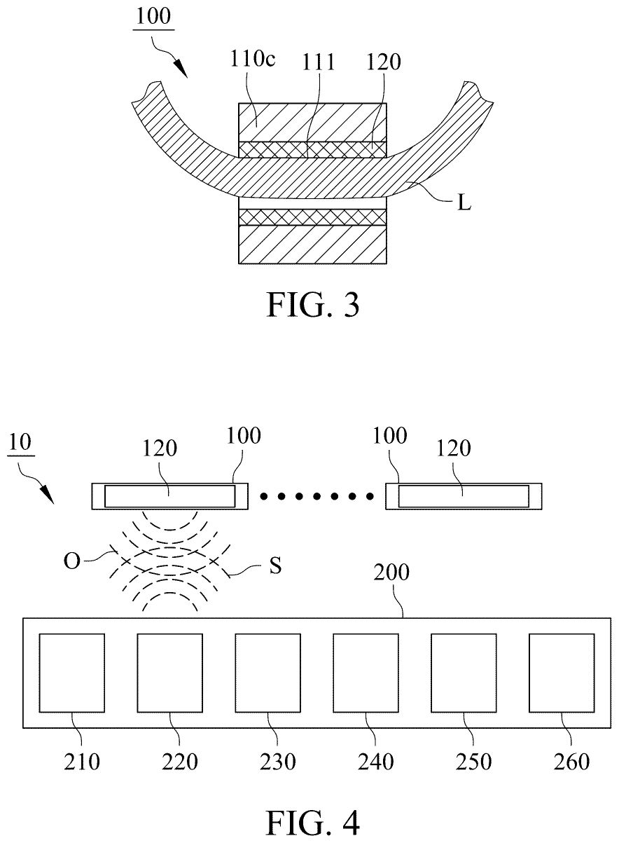

With reference to FIGS. 1 and 2, in this embodiment, the carrier 110 has a body 110c where the through hole 111 is located on and a coupling column 110d which can couple with a base 130 (e.g. suture anchor) planted in the bone. The coupling column 110d is designed to be inserted into a coupling hole 131 of the base 130 to connect the carrier 110 with the base 130. In other embodiments, the base 130 is not necessary and the coupling column 110d of the carrier 110 can be inserted into the bone.

With reference to FIGS. 1 and 4, a sensing system 10 for surgical suture thread includes multiple sensing devices 100 and one wireless reading device 200 mentioned above. Resonance frequency ranges of the wireless sensors 120 in the sensing devices 100 are different and not overlapping, and there are resonance frequencies in each of the resonance frequency ranges. The wireless reading device 200 includes a controller 210, an inductive coil 220, a reader 230 and a computer 240, preferably, the wireless reading device 200 further includes a communicator 250 and a display 260.

Referring to FIG. 4, the controller 210 is provided to generate and transmit oscillation signals S to the inductive coil 220. Oscillation frequency ranges of the oscillation signals S are not overlapping. Each of the oscillation signals S includes multiple oscillation sub-signals with different oscillation frequencies, the oscillation sub-signals are transmitted to the inductive coil 220 to active the wireless sensor 120 in each of the sensing devices 100. As shown in FIG. 6, in this embodiment, the oscillation signals S are a first oscillation signal S1 having a first oscillation frequency range T1 and a second oscillation signal S2 having a second oscillation frequency range T2, the first oscillation signal S1 includes first oscillation sub-signals S11 to S20, and the second oscillation signal S2 includes second oscillation sub-signals S21 to S30. But the number of the oscillation signals S and the oscillation sub-signals are not limited in the present invention. While the oscillation frequency of one of the oscillation sub-signals of one of the oscillation signals S is consistent with one of resonance frequencies within the resonance frequency range of the wireless sensor 120 of one of the sensing devices 100, a resonance frequency signal O is generated by the wireless sensor 120. The reader 230 is provided to receive the resonance frequency signal O and read a phase value corresponding to the oscillation frequency signal O. The computer 240 is provided to receive and convert the phase value to a pressure signal. The pressure signal is transmitted to the display 260 by the communicator 250, and a pressure value of the pressure signal is shown on the display 260. Thus, medical personnel can confirm the force applied to the soft tissue by surgical suture thread L is appropriate or not. Preferably, the pressure signal is transmitted from the communicator 250 to the display 260 via wireless transmission.

With reference to FIGS. 1, 4 and 5, in a sensing method of the present invention, the sensing system 10 including multiple sensing devices 100 and one wireless reading device 200 is provided firstly. A first sensing device 100a and a second sensing device 100b are used in the sensing method of a preferred embodiment of the present invention. The present invention does not restrict the amount of the sensing devices 100, more than two sensing devices can be used based on different requirements in other embodiments.

With reference to FIG. 5, in this embodiment, the first and second sensing devices 100a and 100b are planted onto the bone firstly. The first sensing device 100a includes a first carrier 110a and a first wireless sensor 120a which is mounted in a first through hole 111a of the first carrier 110a. The second sensing device 100b includes a second carrier 110b and a second wireless sensor 120b which is mounted in a second through hole 111b of the second carrier 110b.

With reference to FIGS. 5 and 6, there are first resonance frequencies R11 to R20 in a first resonance frequency range R1 of the first wireless sensor 120a, and there are second resonance frequencies R21 to R20 in a second resonance frequency range R2 of the second wireless sensor 120b. The first resonance frequency range R1 is not overlapping with the second resonance frequency range R2. While the surgical suture thread L is inserted through the soft tissue (e.g. tendon), passed through the first through hole 111a of the first carrier 110a and/or the second through hole 111b of the second carrier 110b and pulled tight, the first wireless sensor 120a is pressured by the surgical suture thread L to generate a resonance frequency which is one of the first oscillation frequencies R11 to R20 within the first resonance frequency range R1, and/or the second wireless sensor 120b is pressured by the surgical suture thread L to generate a resonance frequency which is one of the second resonance frequencies R21 to R30 within the second resonance frequency range R2.

With reference to FIGS. 4 to 6, the controller 210 in the wireless reading device 200 is provided to generate oscillation signals S whose numbers can be varied according to how many sensing devices 100 are used. In this embodiment, the controller 210 is provided to generate and transmit a first oscillation signal S1 and a second oscillation signal S2 to the inductive coil 220. And the controller 210 can generate more than two oscillation signals in other embodiments. The first oscillation signal S1 having a first oscillation frequency range T1 includes first oscillation sub-signals S11 to S20 with different oscillation frequencies, and the second oscillation signal S2 having a second oscillation frequency range T2 includes second oscillation sub-signals S21 to S30 with different oscillation frequencies. And the first oscillation frequency range T1 is not overlapping with the second oscillation frequency range T2.

Referring to FIGS. 5 to 7, next, the first sensing device 100a or the second sensing device 100b is activated, and the activation includes six steps 301 to 306. In a first step 301 of first activation, the first oscillation sub-signals S11 to S20 of the first oscillation signal S1 are provided to activate the first sensing device 100a in order. While the oscillation frequency of one of the first oscillation sub-signals S11 to S20 is consistent with one of the first resonance frequencies R11 to R20 of the first wireless sensor 120a, a first resonance frequency signal O1 is generated by the first wireless sensor 120a. If the oscillation frequencies of the first oscillation sub-signals S11 to S20 are not consistent with any one of the first resonance frequencies R11 to R20 of the first wireless sensor 120a, the second oscillation sub-signals S21 to S30 of the second oscillation signal S2 are provided to activate the first sensing device 100a one by one, and the first resonance frequency signal O1 is generated by the first wireless sensor 120a while the oscillation frequency of one of the second oscillation sub-signals S21 to S30 is consistent with one of the first resonance frequencies R11 to R20 of the first wireless sensor 120a.

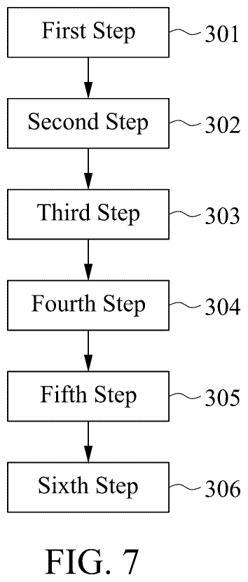

Referring to FIGS. 6 and 7, in a second step 302 of first reading, the reader 230 reads a first phase value corresponding to the first resonance frequency signal O1. In a third step 303 of first computation, the computer 240 converts the first phase value to a first pressure signal and shows a first pressure value of the first pressure signal on the display 260. In this embodiment, the computer 240 transmits the first pressure signal to the display 260 via the communicator 250 to show the first pressure value of the first pressure signal on the display 260. Accordingly, medical personnel can know the force applied to the soft tissue by the surgical suture thread L is proper or not.

Referring to FIGS. 6 and 7, in a fourth step 304 of second activation, the second sensing device 100b is activated by the second oscillation sub-signals S21 to S30 of the second oscillation signal S2 while the oscillation frequency of one of the first oscillation sub-signals S11 to S20 is consistent with one of the first resonance frequencies R11 to R20 of the first wireless sensor 120a, and a second resonance frequency signal O2 is generated by the second wireless sensor 120b as the oscillation frequency of one of the second oscillation sub-signals S21 to S30 is consistent with one of the second resonance frequencies R21 to R30 of the second wireless sensor 120b. On the other hand, the second sensing device 100b is activated by the first oscillation sub-signals S11 to S20 of the first oscillation signal S1 while the oscillation frequency of one of the second oscillation sub-signals S21 to S30 is consistent with one of the first resonance frequencies R11 to R20 of the first wireless sensor 120a, and the second resonance frequency signal O2 is generated by the second wireless sensor 120b while the oscillation frequency of one of the first oscillation sub-signals S11 to S20 is consistent with one of the second resonance frequencies R21 to R30 of the second wireless sensor 120b.

With reference to FIGS. 6 and 7, the reader 230 reads a second phase value corresponding to the second resonance frequency signal O2 in a fifth step 305 of second reading. The computer 240 converts the second phase value to a second pressure signal in a sixth step 306 of second computation, and a second pressure value of the second pressure signal is shown on the display 260. Thus, medical personnel can know whether the force applied to the soft tissue by the surgical suture thread L is appropriate.

During soft tissue suture using the surgical suture thread L which is passed through the through hole 111 on the sensing device 100, the pressure value of the wireless sensor 120 mounted in the through hole 111 and pressured by the surgical suture thread L can be detected by the wireless reading device 200 to confirm whether the force applied to the soft tissue by the surgical suture thread L is appropriate. The tension in the surgical suture thread L can be determined objectively, not according to personal experience only, thereby improving patient comfortableness after surgery. Furthermore, the wireless reading device 200 can be provided to read the information from the sensing device 100 in patient to know the tension of the surgical suture thread L after surgery.

While this invention has been particularly illustrated and described in detail with respect to the preferred embodiments thereof, it will be clearly understood by those skilled in the art that is not limited to the specific features shown and described and various modified and changed in form and details may be made without departing from the scope of the claims.

What is claimed is:

1. A sensing device configured to be planted onto a bone to sense a tension in a surgical suture thread, comprising:
a carrier; and
a wireless sensor mounted in a through hole of the carrier, the wireless sensor is configured to be contacted by the surgical suture thread passed through the through hole and configured to be activated by a wireless reading device to generate a resonance frequency signal.

2. The sensing device in accordance with claim 1, wherein the carrier includes a body and a coupling column, and the through hole of the carrier is located on the body.

3. A sensing system comprising:
a plurality of sensing devices configured to be planted onto a bone to sense a tension in a surgical suture thread, each of the plurality of sensing devices includes a carrier and a wireless sensor, the wireless sensor is mounted in a through hole of the carrier and configured to be contacted by the surgical suture thread passed through the through hole, a resonance frequency range of the wireless sensor of each of the plurality of sensing devices is different and not overlapping; and
a wireless reading device including a controller, an inductive coil, a reader and a computer, the controller is configured to generate a plurality of oscillation signals to the inductive coil, oscillation frequency ranges of the plurality of oscillation signals are not overlapping, each of the plurality of oscillation signals includes a plurality of oscillation sub-signals with different oscillation frequencies, the plurality of oscillation sub-signals are configured to be transmitted to the inductive coil to activate the wireless sensor of each of the plurality of sensing devices, wherein while the oscillation frequency of one of the plurality of oscillation sub-signals of one of the plurality of oscillation signals is consistent with one of a plurality of resonance frequencies within the resonance frequency range of the wireless sensor of one of the plurality of sensing devices, the wireless sensor is configured to generate a resonance frequency signal, the reader is configured to read a phase value corresponding to the resonance frequency signal, and the computer is configured to receive and convert the phase value to a pressure signal.

4. The sensing system in accordance with claim 3, wherein the wireless reading device further includes a communicator which is configured to transmit the pressure signal to a display, and the display is configured to show a pressure value of the pressure signal.

5. The sensing system in accordance with claim 4, wherein the pressure signal is configured to be transmitted from the communicator to the display via wireless transmission.

6. The sensing system in accordance with claim 3, wherein the carrier includes a body and a coupling column, and the through hole of the carrier is located on the body.

7. A sensing method for sensing a force applied to a soft tissue by a surgical suture thread comprising:
providing a first sensing device and a second sensing device which are configured to be planted onto a bone, the first sensing device includes a first carrier and a first wireless sensor which is mounted in a first through hole of the first carrier, the second sensing device includes a second carrier and a second wireless sensor which is mounted in a second through hole of the second carrier, a first resonance frequency range of the first wireless sensor is not overlapping with a second resonance frequency range of the second wireless sensor, the first wireless sensor is configured to be contacted by the surgical suture thread passed through the first through hole to generate a first resonance frequency within the first resonance frequency range, and the second wireless sensor is configured to be contacted by the surgical suture thread passed through the second through hole to generate a second resonance frequency within the second resonance frequency range;
providing a wireless reading device including a controller, an inductive coil, a reader and a computer, the controller is configured to generate a first oscillation signal and a second oscillation signal to the inductive coil, the first oscillation signal includes a plurality of first oscillation sub-signals with different oscillation frequencies within a first oscillation frequency range of the first oscillation signal, the second oscillation signal includes a plurality of second oscillation sub-signals with different oscillation frequencies within a second oscillation frequency range of the second oscillation signal, and the first oscillation frequency range is not overlapping with the second oscillation frequency range; and
activation of the first sensing device and/or the second sensing device including:
a first step of activating the first sensing device by the plurality of first oscillation sub-signals or by the plurality of second oscillation sub-signals, wherein the first wireless sensor of the first sensing device activated by the plurality of first oscillation sub-signals is configured to generate a first resonance frequency signal while the oscillation frequency of one of the plurality of first oscillation sub-signals is consistent with the first resonance frequency of the first wireless sensor, wherein the first sensing device is configure to be activated by the plurality of second oscillation sub-signals while the oscillation frequency of each of the plurality of first oscillation sub-signals is not consistent with the first resonance frequency of the first wireless sensor, and wherein the first wireless sensor of the first sensing device activated by the plurality of second oscillation sub-signals is configured to generate the first resonance frequency signal while the oscillation frequency of one of the plurality of second oscillation sub-signals is consistent with the first resonance frequency of the first wireless sensor;
a second step of reading a first phase value corresponding to the first resonance frequency signal by the reader;
a third step of converting the first phase value to a first pressure signal by the computer and showing a first pressure value of the first pressure signal on a display;
a fourth step of activating the second sensing device by the plurality of second oscillation sub-signals or by the plurality of first oscillation sub-signals, wherein the second sensing device is configured to be activated by the plurality of second oscillation sub-signals while the oscillation frequency of one of the first oscillation sub-signals is consistent with the first resonance frequency of the first wireless sensor, wherein the second wireless sensor of the second sensing device activated by the plurality of second oscillation sub-signals is configured to generate a second resonance frequency signal while the oscillation frequency of one of the plurality of second oscillation sub-signals is consistent with the second resonance frequency of the second wireless sensor, wherein the second sensing device is configured to be activated by the plurality of first oscillation sub-signals while the oscillation frequency of one of the second oscillation sub-signals is consistent with the first resonance frequency of the first wireless sensor, and wherein the second wireless sensor of the second sensing device activated by the plurality of first oscillation sub-signals is configured to generate the second resonance frequency signal while the oscillation frequency of one of the plurality of first oscillation sub-signals is consistent with the second resonance frequency of the second wireless sensor;
a fifth step of reading a second phase value corresponding to the second resonance frequency signal by the reader; and
a sixth step of converting the second phase value to a second pressure signal by the computer and showing a second pressure value of the second pressure signal on the display.

8. The sensing method in accordance with claim 7, wherein the wireless reading device further includes a communicator which is configured to transmit the first and second pressure signals to the display via wireless transmission.
